(19) United States
(12) Patent Application Publication
(10) Pub. No.: US 2024/0307237 A1
(43) Pub. Date: Sep. 19, 2024

(54) TAMPON AND APPLICATOR AND MANUFACTURING RELATED THERETO

(71) Applicant: Edgewell Personal Care Brands, LLC, Chesterfield, MO (US)

(72) Inventors: Joseph Garthaffner, Richmond, VA (US); Michael Burt, Dover, DE (US); Christopher O'Kelly, Richmond, VA (US); Pankaj Nigam, Allendale, NJ (US)

(73) Assignee: Edgewell Personal Care Brands, LLC, Chesterfield, MO (US)

(21) Appl. No.: 18/546,985

(22) PCT Filed: Feb. 17, 2022
(86) PCT No.: PCT/US2022/016753

§ 371 Date: Aug. 18, 2023

Related U.S. Application Data
(60) Provisional application No. 63/151,258, filed on Feb. 19, 2021.

(51) Int. Cl. A61F 13/26; A61F 13/15; A61F 13/20

(52) U.S. Cl. CPC A61F 13/266; A61F 13/2068; A61F 2013/15406; A61F 2013/15471

(57) ABSTRACT

A tampon pledget having a unique forming process enables a reduction in material (by weight) without reducing gram per gram absorbency and optionally improving absorbency. The tampon pledget is formed by a unique carding, cross-lapping, and needle-punching process. A tampon applicator is formed having one or more components from at least one of a sustainable material and a compostable material. A tampon applicator plunger has at least one deformed end. A tampon applicator plunger has improved strength. A tampon assembly has an improved retention force.

### CROSS-REFERENCE TO RELATED APPLICATIONS

This application claims priority to U.S. Provisional Patent Application No. 63/151,258, filed Feb. 19, 2021, the content of which is incorporated herein for reference in its entirety.

### BACKGROUND OF THE INVENTION

### 1. Technical Field

The present disclosure relates to tampon pledgets and tampon applicators, individually and functioning together as a tampon applicator assembly.

### 2. Background Information

Tampon pledgets provide absorbent material for storing menstrual fluids. Absorbent material includes cotton fibers and rayon fibers, where rayon fibers have historically been capable of greater absorbent capacity, that is, the ability to hold a greater amount of fluid than cotton fibers. Rayon fibers are treated and in the eyes of some consumers, causes concerns due to their chemical composition. As such, there is a need for cotton tampon pledgets that are capable of performing as well as rayon tampon pledgets. As such, there is a need to reduce the amount of rayon fibers used by creating improved ways for manufacturing and forming tampon pledgets to maintain or increase absorbency.

Tampon applicators have traditionally been made from either plastic or cardboard. Plastic applicators have been perceived by some consumers as having improved comfort, and thus the vast majority of consumers of tampon applicator products utilize plastic applicators. Notwithstanding, as the world becomes further aware and keen to mitigate against the harms that non-sustainable, non-recyclable, non-biodegradable, and non-compostable products pose, there is a further need for better performing naturally based applicators as well as those that are more environmentally friendly in general.

### SUMMARY OF THE INVENTION

According to an aspect of the present invention, a tampon pledget is provided. The tampon pledget includes absorbent fibers such as rayon or cotton or combinations thereof. The tampon pledget is constructed of a web, loose fibers, or multiple webs that are stacked upon each other such as in (but not limited to) a cross-pad configuration or an offset configuration. The tampon pledget material (absorbent fibers) has been constructed from material that was carded, crosslapped, and needle-punched. The absorbent fiber has been needle-punched such that there are over 5 needles per square centimeter of the absorbent material, or over 20 needles per square centimeter, or over or equal to 30 needles per square centimeter, or over or equal to 40 needles per square centimeter, or equal to or over 70 needles per square centimeter, or up to or equal to 150 needles per square centimeter, or between about 5 needles per square centimeter and about 150 needles per square centimeter, or between about 40 needles per square centimeter and about 140 needles per square centimeter, or between 70 needles per square centimeter and 130 needles per square centimeter, or between 80 and 120 needles per square centimeter, or between about 30 needles per square centimeter and about 90 needles per square centimeter, or about 40 needles per square centimeter, or about 80 needles per square centimeter. The tampon pledget thereafter is formed by compressing the absorbent material by any of axial and/or radial compression to form a generally cylindrical shape that optionally has a tapered tip. A string is provided in the removal end of the pledget to accommodate easier removal for the consumer.

The tampon pledget of the present disclosure has a reduced mass with respect to its respective existing tampons in a given absorbency band (e.g. a Regular tampon absorbs 6 grams to 9 grams of fluid, a Super tampon absorbs 9 grams to 12 grams of fluid, an Super Plus tampon absorbs 12 grams to 15 grams of fluid, an Ultra tampon absorbs 15 grams to 18 grams of fluid, and a Light tampon absorbs up to 6 grams of fluid) yet is able to absorb as much or more fluid than such similarly classified tampons in that absorbency band. In certain embodiments, the tampon pledget according the present disclosure includes a lower basis weight yet is able to absorb over 10% or more fluid, or over 15% or more fluid, or over 18% or more fluid than known tampons in its respective absorbency band. In one embodiment of the present disclosure, a tampon pledget according to the present disclosure has a lower density than currently available tampons in its respective absorbency band. In one embodiment of the present disclosure, a tampon pledget according to the present disclosure contains less mass than tampon pledgets currently available in its respective absorbency band.

According to another aspect of the present disclosure, a tampon applicator system is provided. The tampon applicator system includes materials that are compostable material, a biodegradable material, a sustainable material, a natural material, a recycled material, and a recyclable material is provided. In one embodiment, the applicator barrel is one of the aforementioned materials. In one embodiment, the applicator plunger is one of the aforementioned materials. In one embodiment, the applicator barrel and applicator plunger are one of the aforementioned materials. In one embodiment, the applicator barrel and applicator plunger are different materials (but included one of the aforementioned materials).

In a further aspect of the present disclosure, the tampon applicator system (including the tampon pledget) are compostable materials, biodegradable materials, sustainable materials, natural materials, recycled materials, and recyclable materials. For example, in one embodiment, a cotton tampon pledget is provided (i.e., a natural material), a bioplastic applicator barrel (i.e., a natural material) is provided, and a cardboard plunger is provided (i.e. a compostable material). In other embodiments, a recycled plastic material is used in the applicator system, either the applicator barrel or the applicator plunger, or both.

In a further aspect of the present disclosure, an insertion end of the plunger is provided. The plunger is generally cylindrical having an insertion end, a consumer end substantially opposite the insertion end, and a middle portion separating and connecting the insertion end and the consumer end. The insertion end has a deformed end creating at least three vertices on the end of the plunger. The deformed plunger end has up to ten vertices. The deformed plunger end is a polygon, for instance, a triangle, quadrilateral, pentagon, hexagon, heptagon octagon nonagon, decagon, etc. The diameter of the polygon is between about 9.25 mm and about 10.25 mm, and more preferably 9.5 mm to about 10 mm, or about 9.75 mm.

The tampon applicator assembly has additional preferred characteristics. For instance, the tampon applicator assembly has an improved ejection force (i.e., the force the user must apply to the consumer end of the plunger to push it inward in the applicator barrel and thereby pushing the withdrawal end of the pledget, where the insertion end of the pledget pushes through the insertion end of the applicator barrel enabling the pledget to be ejected from the applicator barrel by way of a full stroke of movement of the plunger). For instance, the plunger has an improved column strength. For instance, the plunger with the deformed insertion end has an improved retention force over prior art plungers.

Some tampons are available in a format that includes an applicator to facilitate insertion of the tampon. There are a variety of different types of tampon applicators available. For example, some types of applicators include a barrel portion and a plunger. Prior to use, the tampon is disposed within an interior cavity of the barrel. The plunger is operable to move relative to the barrel to expel the tampon during insertion. The plunger is inserted into the applicator barrel and the ends of the plunger are formed thereafter. In embodiments having a compact plunger including two portions—an inner plunger and an outer plunger—the inner plunger is assembled into the outer plunger prior to insertion into the tampon applicator barrel. The tampon is thereafter inserted through the insertion end.

The aforementioned aspects of the invention include unique manufacturing and assembly contemplated further herein, including unique apparatus' for constructing aspects of the present disclosure.

The present method and advantages associated therewith will become more readily apparent in view of the detailed description provided below, including the accompanying drawings.

### BRIEF DESCRIPTION OF THE DRAWINGS

FIG. 1 is a cross-sectional photo of multi-lobal rayon fibers;

FIG. 2 is a cross-sectional photo of single lobed rayon fibers;

FIG. 3 is a cross-sectional photo of cotton fibers;

FIG. 4 is a photo of a tampon according to the present disclosure;

FIG. 5 is a chart showing tampon pledget absorbency data of cotton fibers and multi-lobal rayon fibers;

FIG. 6 is a chart showing gram per gram absorbency data for cotton fibers and multi-lobal fibers;

FIG. 7 is a chart describing process steps for making a tampon pledget according to the present disclosure;

FIG. 8 is a chart describing process steps for making a tampon pledget according to the present disclosure;

FIG. 9 is a chart describing process steps for making a tampon pledget according to the present disclosure;

FIG. 10 is a schematic of an exemplary cross-pad lay-up for a tampon pledget according to the present disclosure;

FIG. 11 is a schematic of an exemplary tampon pledget forming process according to the present disclosure;

FIG. 12 is a process diagram contemplating the tampon pledget form process according to the present disclosure;

FIGS. 13-19 are diagrammatic views of exemplary embodiments of an end of a tampon applicator plunger according to the present disclosure;

FIG. 20 is a photo of tampon applicator plungers prior to undergoing the forming process according to the present disclosure;

FIG. 21 is a photo of exemplary tampon applicator plungers after undergoing the forming process according to the present disclosure;

FIG. 22 is a diagrammatic representation of an exemplary forming tool and formed tampon applicator plunger according to the present disclosure;

FIG. 23 is an angled top view of an exemplary forming tool according to the present disclosure;

FIG. 24 is a side elevation sectional view of an exemplary forming tool according to the present disclosure;

FIGS. 25-26 are schematic views of exemplary forming tools according to the present disclosure;

FIG. 27 is a schematic of an exemplary tampon applicator forming machine according to the present disclosure;

FIG. 28 is a schematic of an exemplary tampon applicator forming machine according to the present disclosure;

FIG. 29 is a cross-sectional view of an exemplary tampon applicator assembly according to the present disclosure;

FIG. 30 is a cross-sectional view of an exemplary tampon applicator assembly according to the present disclosure; and

FIG. 31 is a cross-sectional view of an exemplary tampon applicator assembly according to the present disclosure.

### DETAILED DESCRIPTION

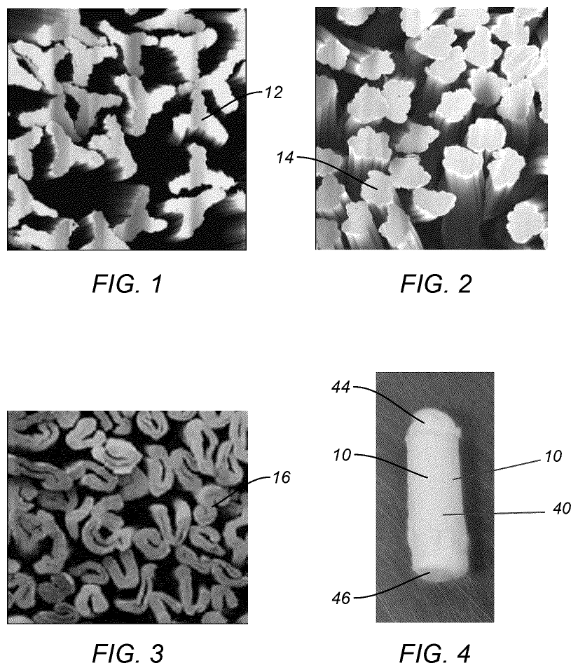

As shown by FIGS. 1-3, various absorbent fibers are utilized in forming a tampon pledget 10. For simplicity, the terms “pledget”, “tampon” 10, and “tampon pledget” 10 can be used interchangeably for the purposes of the present disclosure, and is generally shown in FIG. 4. FIGS. 1-3 show cross-sectional shapes of fibers obtained from a scanning electron microscope. FIG. 1 shows a multi-lobal rayon fiber 12 such as those sold under the tradename GALAXY® by Kelheim. FIG. 2 shows a single-lobal (or staple) rayon fiber 14 such as those sold by Kelheim or Lenzing. FIG. 3 shows a cotton fiber 16 such as from Barnhardt.

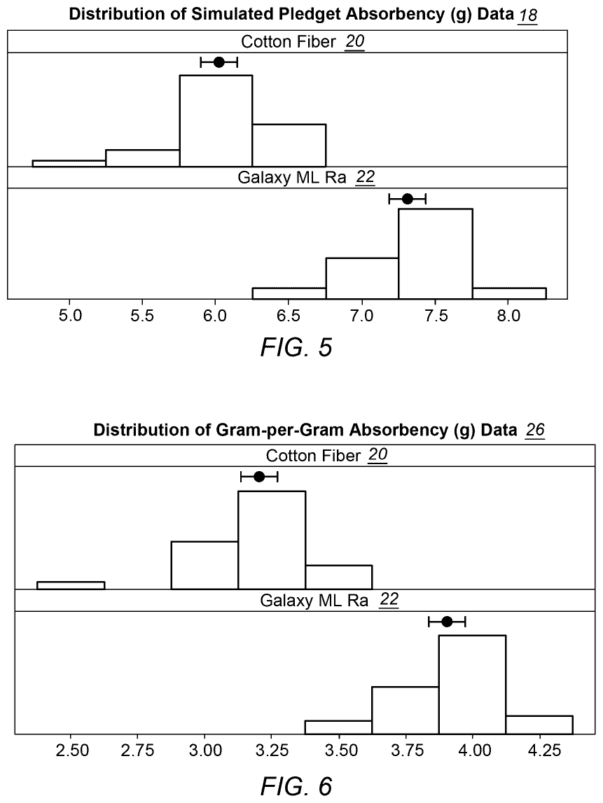

In general, multi-lobal fibers 12 tend to have the greatest surface area and thus the greatest absorbent potential. Test data suggests this to be true, including the tables shown in FIGS. 5 and 6. Indeed, FIG. 5 titled “Distribution of Simulated Pledget Absorbency (g) Data” (and noted as reference numeral 18) demonstrates that multi-lobal rayon fiber 22 has a higher theoretical regular pledget absorbency of about 7.5 g (and between about 6.2 g and about 8.3 g) than cotton fiber 20 that has an absorbency of about 6.0 g (and between about 4.7 g and about 6.7 g). Similarly, FIG. 6 titled “Distribution of Gram-per-Gram Absorbency Data” (and noted as reference numeral 26) describes a distribution of gram per gram absorbency of fibers, where the multilobal rayon fiber 12 has an average absorbency just under 4.0 g/g (with a range of about 3.3 g/g to about 4.3 g/g), while the cotton fiber 16 has an average absorbency of just under 3.25 g/g (with a range of between about 2.3 g/g to about 3.6 g/g).

Absorbency data throughout the present disclosure has been determined in accordance with FDA requirements, and uses an experimental set-up (syngyna test chamber) as per FIG. 1 and FIG. 2 of 21 C.F.R. Part 801.432(f)(2). In the absorbency test, an unlubricated condom, with tensile strength between 17 Mega Pascals (MPa) and 30 MPa, as measured according to the procedure in the American Society for Testing and Materials (ASTM) D 3492-97, “Standard Specification for Rubber Contraceptives (Male Condoms)” ¹ for determining tensile strength, which is incorporated by reference in accordance with 5 U.S.C. 552(a), is attached to the large end of a glass chamber (or a chamber made from hard transparent plastic) with a rubber band (see FIG. 1) and pushed through the small end of the chamber using a smooth, finished rod. The condom is pulled through until all slack is removed. The tip of the condom is cut off and the remaining end of the condom is stretched over the end of the tube and secured with a rubber band. A pre-weighed (to the nearest 0.01 gram) tampon is placed within the condom membrane so that the center of gravity of the tampon is at the center of the chamber. An infusion needle (14 gauge) is inserted through the septum created by the condom tip until it contacts the end of the tampon. The outer chamber is filled with water pumped from a temperature-controlled water bath to maintain the average temperature at 27+/−1 degree Celsius. The water returns to the water bath as shown in FIG. 2. Syngyna fluid (10 grams sodium chloride, 0.5 gram Certified Reagent Acid Fushsin, 1,000 milliliters distilled water) is then pumped through the infusion needle at a rate of 50 milliliters per hour. The test shall be terminated when the tampon is saturated and the first drop of fluid exits the apparatus. (The test result shall be discarded if fluid is detected in the folds of the condom before the tampon is saturated). The water is then drained and the tampon is removed and immediately weighed to the nearest 0.01 gram. The absorbency of the tampon is determined by subtracting its dry weight from this value. The condom shall be replaced after 10 tests or at the end of the day during which the condom is used in testing, whichever occurs first.

Surprisingly, the applicants of the present disclosure have achieved a process and resultant tampon pledget 10 that enables parity or improved pledget absorbency with fewer fibers and thus less mass. This is preferable for many reasons, namely the ability to use fewer fibers and thus reduce waste and additional impact on the environment (i.e., energy to make the fibers, transport the fibers, etc.), to use less absorbent that have fewer additives or are natural, or to avoid adding additional natural fibers to compensate for their lower absorbent potential. If a greater number of natural fibers are required to provide the appropriate amount of absorbency, the pledget 10 either becomes larger or has to be compressed further to maintain the size of a pledget 10 with more absorbent fibers. Adding fibers adds costs and where the pledget 10 size cannot be larger, additional fibers can work against absorption due to over densification resulting in the inability for fibers to fully-expand and reach their absorbent potential.

Surprisingly, the gram per gram absorbency of the tampon pledget 10 of the present disclosure is parity (despite the use of less absorbent fibers) or is improved (despite the use of less absorbent fibers, or fewer fibers). The gram per gram absorbency the is the maximum volume of fluid that the tampon pledget 10 absorbs divided by the mass of the dry tampon pledget 10 (prior to absorption).

In one aspect of the present disclosure, a pledget 10 has at least 10% less mass than existing tampon pledgets sold under the brand PLAYTEX GENTLE GLIDE®, or at least 15% less mass, or at least 16% less mass, or at least 18% less mass. In one aspect of the present disclosure, a cotton 14 pledget 10 has at least 10% less mass than a pledget 10 made from rayon fibers 12, 16, or at least 15% less mass than a pledget 10 made from rayon fibers 12, 16, or at least 16% less mass than a pledget 10 made from rayon fibers, 12, 16.

In one aspect of the present disclosure, a pledget 10 has at least 10% more absorbency than tampon pledgets sold under the brand PLAYTEX GENTLE GLIDE®, or at least 15% more absorbency, or at least 16% more absorbency, or at least 18% more absorbency. In one aspect of the present disclosure, a cotton 14 pledget 10 has at least 10% or more absorbency than a pledget 10 made from rayon fibers 12, 16, or at least 15% more absorbency than a pledget 10 made from rayon fibers 12, 16, or at least 16% more absorbency than a pledget 10 made from rayon fibers 12, 16.

Fibers have a fiber diameter of about 7 microns to about 40 microns, or about 25 microns to about 40 microns, or about 30 microns to about 40 microns. Fibers have a fiber length of about 0.9 inches (about 2.3 cm) to about 1.2 inches (about 3 cm), or about 0.90 inches (about 2.3 cm) to about 1.0 inches (2.54 cm).

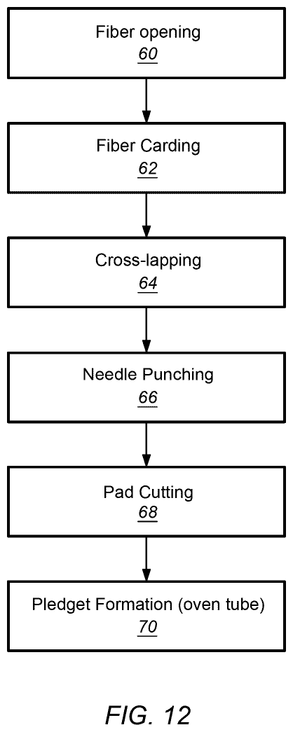

The process diagram in FIG. 12 exemplifies the unique process of the present disclosure. Fiber is brought in bales and undergoes a step of separating the fibers out of the bales (this step is referred to by reference numeral 60). The loose fibers are transported into a carding machine where the fibers are aligned such that they are generally parallel (this step is referred to by reference numeral 62). After carding, the aligned fibers are transferred into a webbing machine where the fibers are crosslapped by layering the fibrous web onto itself (this step is referred to by reference numeral 64). The crosslapping is done such that the fibrous web layers are orthogonal to the immediately adjacent fibrous web layer. After webbing, the fibrous web is needle punched (this step is referred to by reference numeral 66). During the needle-punching step 66, a needle punching die (not shown) provides about forty (40) punches per square centimeter to about eighty (80) punches per square centimeter on the fibrous web. This is in contrast to prior processes where four (4) punches per square centimeter were provided on the fibrous web.

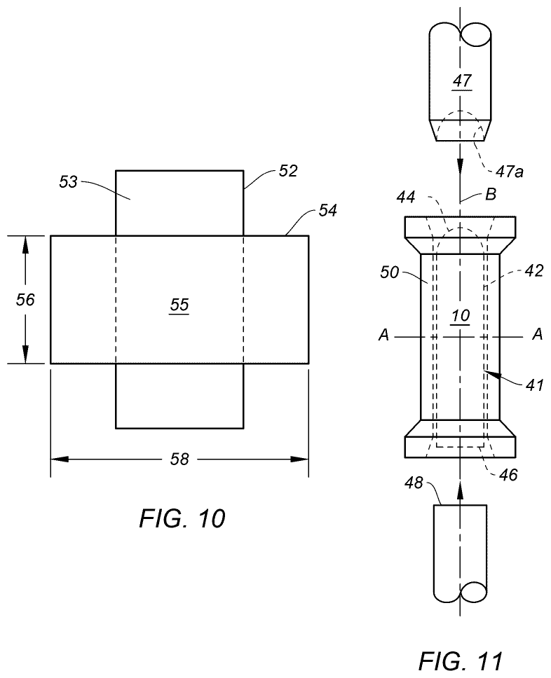

In some forming processes, a cross-pad pledget configuration 53 is provided, which is exemplified in FIG. 10. For a cross-pad configuration 53 for a tampon 10, at least two pads 52, 54 are used where at least one top pad 54 is placed on top of and generally perpendicular to at least one bottom pad 52. The at least one top pad 54 and at least one bottom pad 52 form a cross-pad layup (or cross-pad configuration) 53. While this configuration 53 is generally referred to as a “cross” given the at least one top pad 54 is generally perpendicular placement with respect to the at least one bottom pad 52, other configurations are possible thereby creating additional shapes. Once the cross-pad layup 53 is provided, it is compressed about the center overlapping region 55 of the at least two pads 52, 54 to form a generally cylindrical shape 40. And oven tube 50 and a ram 47, 48 are used to provide the appropriate shape and structure. Additional shaped forming of the insertion end can be achieved by utilizing a ram 47 including a doming part 47a, where it is further useful to heat such doming part 47a. In embodiments utilizing a heated doming tool 47a, the temperature of the tool is about 380 degrees Fahrenheit. The tampon 10 forming equipment includes two cylinders, where the upper cylinder (for ram 47) applies a pressure of between about 65 psi and about 80 psi and the lower cylinder (for ram 48) is between about 75 and about 90 psi. Current cross-pad configuration pledgets (as existing prior to the time of filing the present disclosure) are known in the market under the brand PLAYTEX® SPORT® and PLAYTEX® GENTLE GLIDE®. In other forming processes, a single fibrous pad (not shown) is rolled and thereafter compressed into a generally cylindrical pledget 10. In yet further forming processes, loose fibers are compressed into the form of a tampon 10. In any of the aforementioned forming processes, a cover stock (not shown) can be applied and sealed to itself and/or the pledget 10 by way of heat, ultrasonics, glue, or other known binding processes. Cover stocks (also known as overwraps) are useful to provide additional structure to the pledget 10, control expansion or absorption or retention of fluids.

The tampon pledget 10 of the present disclosure is formed from a web of material (after the opening step (60) the carding step (62), the cross-lapping step (64) and punching process step (66) further described herein), where the web is cut to form a bat or pad of material in a pad-cutting process step (68). The bat or pad 52, 54 has a width 56 of between about one inch (about 2.5 cm) to three inches (about 7.6 cm), or about two inches (about 5.1 cm). Depending on the structure of the tampon pledget 10, a cross-pad configuration 53 has a longest dimension 58 (i.e., a length or width, depending on how it is positioned within the cross-pad configuration 53) of between about three inches (about 7.6 cm) to five inches (about 12.7 cm), or about four inches (about 10.2 inches). The tampon pledget 10 having at least one pad by way of the pad cutting step (68) is then formed (for example by way of an oven tube) by way of process step (70) as further described below.

Surprisingly, it has been found that there is a negative correlation between gram per gram absorbency and density of the pledget 10. Once formed, the pledget 10 of the present disclosure has a density of that is less than the density currently sold under the brand PLAYTEX® GENTLE GLIDE® (as compared respectively amongst pledgets in a specific absorbency range).

The pad lay-up is then compressed by, for example, ramming the pads 52 and 54 into a heated oven tube 50 (FIG. 11) such that the fibers of the pads 52 and 54 are radially compressed in a transverse or width direction along axis A into a generally cylindrical form 40. In one embodiment, an inside diameter of the oven tube 50 is about 0.407 in. for a Super Absorbency type tampon 10, and a target heating temperature is about two hundred thirty degrees Fahrenheit (230° F.). In one embodiment, the cylindrical form 40 is radially compressed in the oven tube 50 at an outer circumference 42 of the cylindrical form 40 along axis A. In accordance with one aspect of the invention, the cylindrical form 41 [of the tampon pledget yet to be fully formed] is then axially compressed in the oven tube 50 in a direction along a vertical axis B. In one embodiment, the form 40 is axially compressed along vertical axis B as pressure is applied at an insertion end 44 and a bottom end 46 of the cylindrical form 40. In one embodiment, the insertion end 44 is axially compressed with a conical/semi-circle shaped portion 47a on ram 47 and the withdrawal end 46 is axially compressed with a flat shaped ram 48. In one embodiment, axial compression of the ends 44 and 46 is performed at a temperature of about three hundred eighty-five degrees Fahrenheit (385° F.) for a dwell time of about twelve seconds (12 sec).

In a further advantageous aspect of the tampon pledget 10 manufacturing process of the present disclosure is that no water is required. Water inherently reduces the absorbent capacity of the fiber as it is absorbed by the fiber, so avoiding the use of water is helpful in promoting end-product performance.

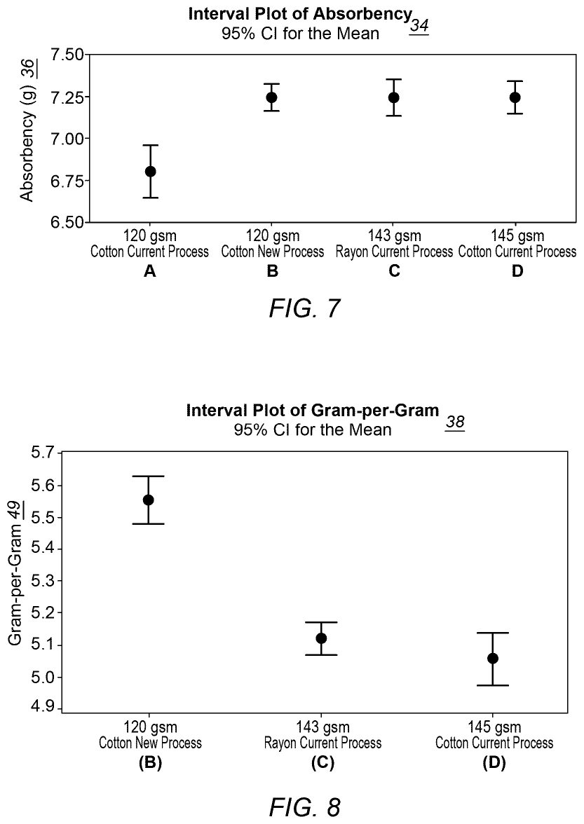

As shown in FIGS. 7-9, the absorbency and gram per gram absorbency of the new sample tampon 10 with a mass of 120 gsm had the highest gram per gram absorbency despite having less than or equal to mass of the other samples made with the control process. For the sake of a more direct comparison, a 100% cotton tampon pledget 10 trial processed with no water in the control process was run. Even with the removal of water from the control process, the new process yielded a tampon 10 that outperformed the modified control process tampon 10 by about a half a gram of absorbency.

For clarity, FIG. 7 describes an Interval Plot of Absorbency 34 comparing Samples A, B, C, and D by way of absorbency (g) 36. FIG. 7 demonstrates data at a 95% confidence interval for mean data reported. Sample A has 100% cotton fibers with a 120 gsm basis weight as formed by the current control process, sample B has 100% cotton fibers with a 120 gsm basis weight as formed by the new process, Sample C is made by 100% multi-lobed rayon fibers with a 143 gsm basis weight as formed by the current process, and Sample D has 100% cotton fibers with a 145 gsm basis weight as formed by the current process. FIG. 8 is an Interval Plot of Gram-per-Gram absorbency 38 of Samples B, C, and D by way of Gram-per-Gram Absorbency (49). As shown in both plots, the tampon 10 of the present disclosure as exemplified by the sample having a 100% cotton fibers with a 120 gsm basis weight formed by the new process outperformed the test pledgets made by the current process, even those with more absorbent fibers and those with more fiber (by way of being a higher basis weight).

To expedite bench testing, tampon pledget 10 construction can be simulated by manually producing a test pledget 10 without the webbing step 64. Cotton fiber 14 such as those sold by Barnhardt as used for some samples and Kelheim Galaxy ML Rayon 16 was used for others, as described further in Table 1 below. To prepare the samples, the test material (cotton 14 or rayon 12, 16) was removed from prepackaged bales and opened by hand to separate the fibers and remove any effect of bale compression. Approximately 1.88 g of fibers was measured and then manually stuffed into a PLAYTX® SPORT® regular absorbency tampon pledget 10 oven tube and thereafter compressed into a further defined shape with a PLAYTX® SPORT® regular absorbency tampon pledget 10 via a ram 47 with doming portion 47a.

TABLE 1
Summary of test pledgets 10 leading
to the results charted in FIGS. 7-8

| Material | Process | Weight | Formation |
|---|---|---|---|
| Multi-lobal Rayon | Control | 143 gsm | With Water |
| Cotton | Control | 145 gsm | No Water |
| Cotton | Control | 120 gsm | No Water |
| Cotton | New | 120 gsm | No Water |

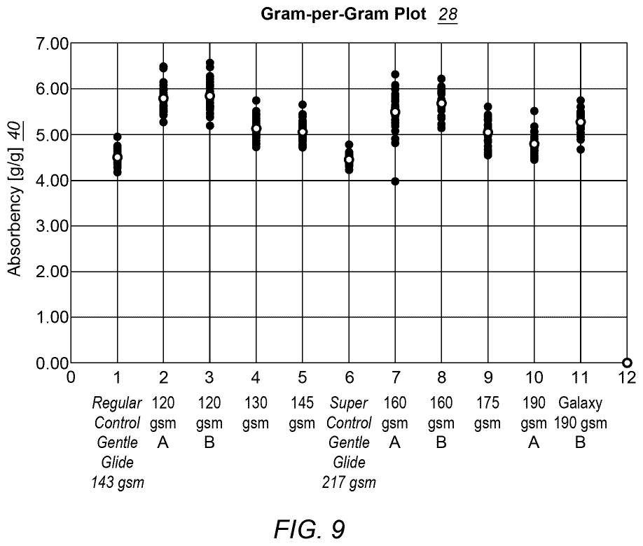

FIG. 9 titled “Gram-per-Gram Plot” (and noted as reference numeral 28) is another plot showing gram-per-gram absorbency 49 of additional embodiments of the present disclosure. As shown, Sample 1 labeled “Regular Control GENTLE GLIDE® 143 gsm” was a control sample, a current GENTLE GLIDE® with regular absorbency, made from rayon fibers with a 143 gsm basis weight. Samples 2-5 are new samples of the present disclosure, including 100% cotton fibers with basis weights of 120 gsm, 120 gsm, 130 gsm, and 145 gsm, respectively. Samples 2 and 3 were separate lots. Sample 6 labeled “Super Control Gentle Glide 217 gsm” was a control sample, a current GENTLE GLIDE® with super absorbency, made from rayon fibers with a 217 g basis weight. Samples 7-10 are new samples of the present disclosure, including 100% cotton fibers with basis weights of 160 gsm, 160 gsm, 175 gsm, and 190 gsm, respectively. Sample 11 is a sample of the present disclosure using 100% Galaxy trilobal rayon fibers, having a basis weight of 190 gsm. As shown, the inventive samples outperformed the control samples with lower basis weights of between about 10 g and about 25 g (for regular absorbency), and of between about 30 g and about 60 g (for super absorbency). Indeed, the tampon of the present disclosure is capable of equal or superior gram-per-gram absorbency with at minimum, about 10 gsm to about 25 gsm lower basis weight; or for a regular absorbency tampon, a basis weight between about 120 gsm and about 145 gsm, or between about 120 gsm and about 140 gsm, or between about 120 gsm and about 130 gsm; or for a super absorbency tampon, a basis weight of between about 160 gsm to about 190 gsm, or between about 160 gsm and about 175 gsm.

Extrapolating the data based on the above trials, it is believed the present disclosure provides the following improved mass (versus currently sold products branded PLAYTEX®):

| Material | Sort | Current GSM | New GSM |
|---|---|---|---|
| Rayon | Sport Regular | 190 | 155 |
| Rayon | Sport Super | 262 | 214 |
| Rayon | Sport Super Plus | 389 | 317 |
| Rayon | Simply Gentle Glide | 144 | 117 |
| | Regular | | |
| Rayon | Simply Gentle Glide | 217 | 177 |
| | Super | | |
| Rayon | Simply Gentle Glide - | 353 | 288 |
| | Super Plus | | |
| Rayon | Simply Gentle Glide; | 438 | 357 |
| | Sport Ultra | | |
| Cotton | Inventive Sample - | N/A | 125 |
| | Regular | | |
| Cotton | Inventive Sample - | N/A | 165 |
| | Super | | |

A further aspect of the present disclosure regards the tampon applicator 120 including the tampon applicator plunger 80. For simplicity, the term “plunger” 80 will be used interchangeably with “applicator plunger” 80 and also “tampon applicator plunger” 80. For further simplicity, the term applicator barrel 122 will refer to the non-plunger 80 portion of the tampon applicator 120. A tampon applicator 120 using at least one of a compostable material, a sustainable material, a natural material, a recycled material, and a recyclable material is provided. In some embodiments, the applicator barrel 122 is a sustainable material, a natural material, and/or a compostable material. In some embodiments, the applicator plunger 80 is a sustainable material and/or a compostable material.

Examples of such materials can include bioplastics, starch-based materials, naturally derived fibrous materials, cellulosic materials, bioplastics, paper, paperboard, cardboard, and bygasse products. Sugar cane, Corn, potato, rice, seaweed, and mushroom-based materials are exemplary of those that may be naturally based, starch-based, and/or cellulosic-based.

In one embodiment, the applicator barrel 122 includes a natural-based material that is optionally starch-based or cellulosic, or is otherwise categorized as a bioplastic. In an embodiment, the plunger 80 is a compostable material such as a paper, paperboard, or cardboard material. Preferably, one or more components of the applicator 120 includes materials other than those of traditional oil-based resins such as low-density polyethylene, polypropylene, and/or polyester.

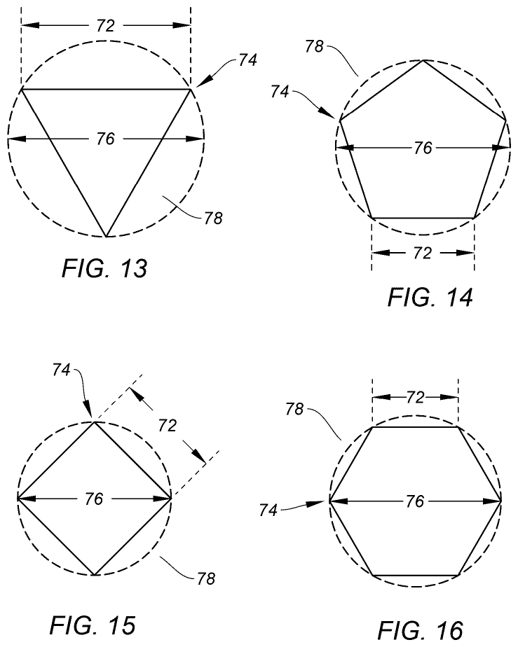

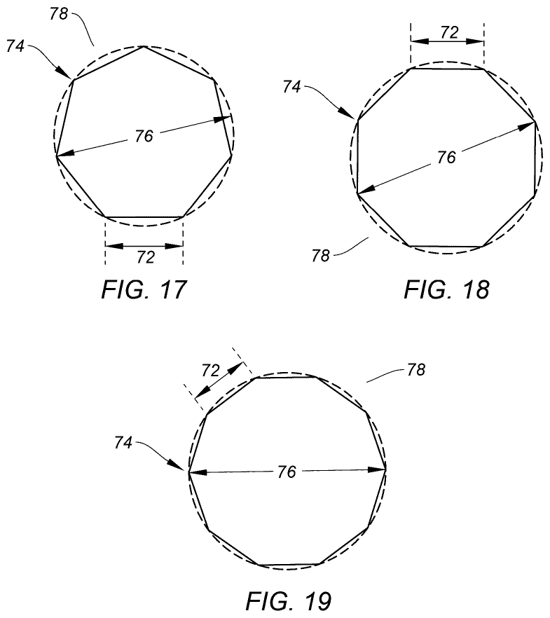

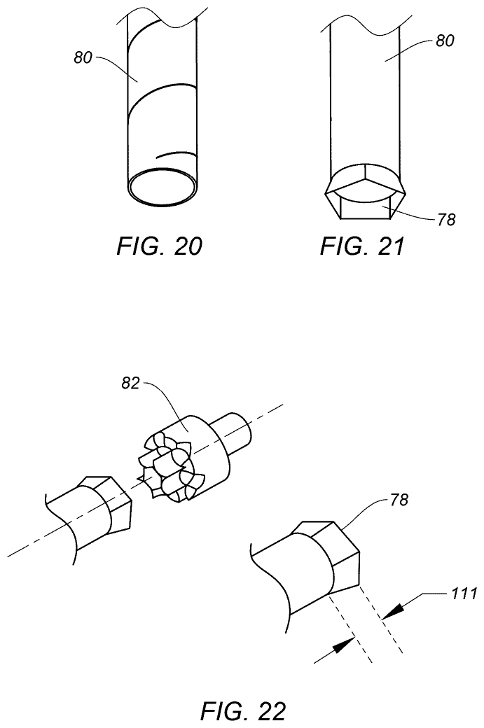

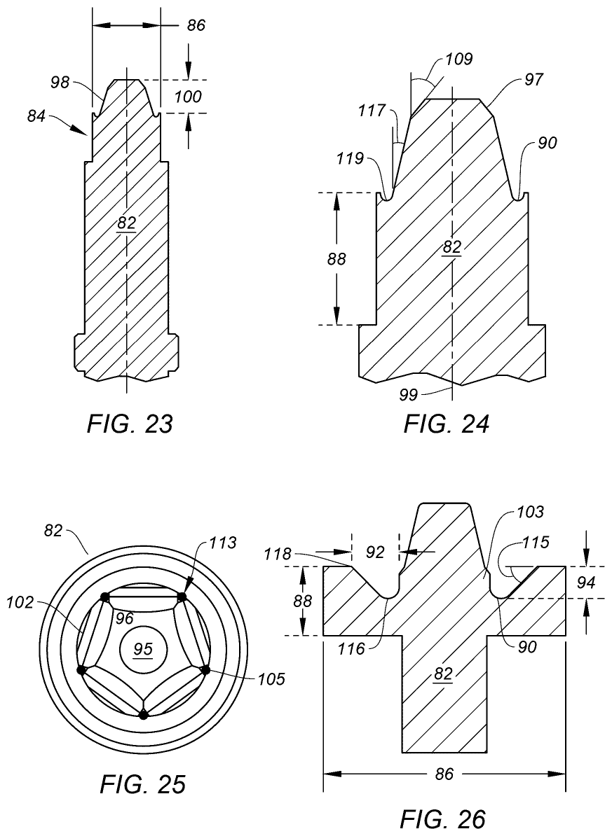

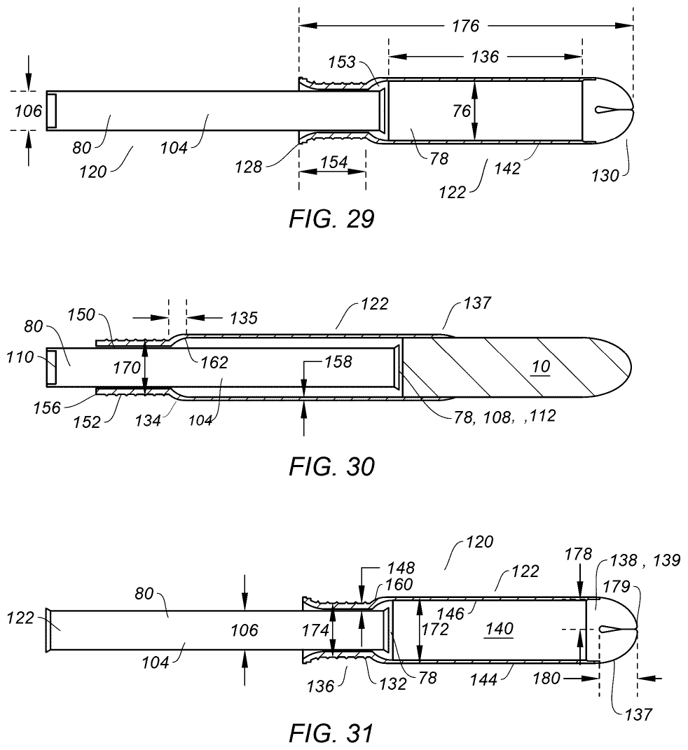

FIGS. 13-31 focus on the unique plunger 80 according to the present disclosure, including the unique plunger 80 formation equipment (FIGS. 22-28), as well as the unique plunger 80 as part of an applicator assembly (FIGS. 29-31). The plunger 80 of the present disclosure has a deformed insertion end 78 that facilitates retention of the plunger 80 in the applicator barrel 122 during use. The deformed insertion end 78 is unlike prior retention features using prongs, rings, or flares. Indeed, the deformed insertion end, utilizing a forming dye, pushes the plunger insertion end outward into a shape having at least three vertices 74 that form a circumscribed circle about the shape. These vertices 74 create a maximum diameter or maximum dimension of the deformed insertion end 78 that is greater than the maximum diameter or dimension 106 of the main body portion 104 of the plunger 80.

The deformed plunger end 78 having the aforementioned vertices 74 can be on at least one of the insertion end 108 and the consumer end 110. In the latter, where a deformed plunger end 112 is on at least the consumer end (or finger end) 110, it provides an enlarged and resilient area to either grip about its periphery or as a finger pad for one's finger to be placed over the consumer end 110.

As exemplified in FIGS. 13-19, the deformed plunger end can be shaped with an even number of vertices 74 or an odd number of vertices. Preferably, there are between 3 and 10 vertices 74. Too few vertices 74 can lead to the inability to adequately retain a plunger 80 within the applicator barrel 122 or can lead to an insufficiently larger or comfortable consumer end 110 of the plunger 80. Too many vertices 74 can be difficult to form and effectively result in a somewhat continuously flared shape, which can lead to a reduced retention force. Furthermore, it can be advantageous to have an odd number of vertices 74 such that there is less symmetry (or no symmetry) along the cross-section of the deformed plunger end 78 (and/or 112), thereby increasing its resiliency in both column strength (or collapse force) and retention force (both properties and related tests described further below). Preferably, the cross-sectional shape of the deformed plunger end 78 (and/or 112) is triangular (where it can be an equilateral triangle), a polygon such as a rhomboid, square, rectangle, kite, trapezoid, pentagon hexagon, heptagon, octagon, enneagon (or nonagon), decagon, etc. In general, interior angles of such deformed plunger end 78 (and/or 112) are between about 60 degrees and about 144 degrees.

In a further aspect of the present disclosure, an insertion end 108 of the plunger 80 is provided. The plunger 80 is generally cylindrical. The insertion end 108 has a deformed end 78 creating at least three vertices 74 on the insertion end 108 and/or consumer end 110 of the plunger 80. The deformed plunger end 78 (and/or 112) has up to ten vertices. The deformed plunger end 78 (and/or 112) is a polygon, for instance, a triangle, quadrilateral, pentagon, hexagon, heptagon octagon nonagon, decagon, etc. In a first embodiment, the diameter 76 of the circumscribed circle (as shown in a dashed line on FIGS. 13-19) about the polygon is between about 9.25 mm and about 10.25 mm, and more preferably 9.5 mm to about 10 mm, or about 9.75 mm. In another embodiment, the diameter 76 of the circumscribed circle (as shown in a dashed line on FIGS. 13-19) about the polygon is between about 6 mm and about 7.25 mm, and more preferably 6.25 mm to about 7 mm, or about 6.5 mm to about 6.9 mm. Deforming the insertion end 108 and/or consumer end 110 of the plunger 80 outside of the taught range can lead to insufficient retention force and/or tearing of the plunger 80.

The side lengths 72 of the deformed plunger end 78 (and/or 112) is between about 1 mm and about 10 mm, or about 3 mm to about 8 mm, or about 4 mm to about 6 mm. The total perimeter of the deformed plunger end is between about 18 mm and about 30 mm, which is substantially similar to the circumference of the non-deformed portion of the plunger. The height 111 of the deformed plunger end is 78, 112, is up to about 5 mm, or between about 0.5 mm and about 5 mm, or between about 0.5 mm and about 3 mm, or between about 2 mm and about 5 mm. The percentage of length of the deformed plunger end with respect to the total plunger length is between about 0.2% and up to about 1%. The percentage of length of the deformed plunger end with respect to the length of the interior surface of the applicator barrel where the deformed plunger end mates is about 50% to 100%.

The deformed plunger end 78 (and/or 112) is superior to prior formed ends in that it yields a better retention force (such that the plunger does not slip-out of the applicator barrel 122 during use which can cause frustrating and unnecessary steps of reinserting the string through the plunger 80 and reinserting the plunger 80 into the applicator barrel 122. The samples of the present disclosure exhibit a retention force of greater than 240 grams, or greater than 245 g, or greater than 250 g, or greater than 275 g, or greater than 300 g, or up to about 325 g, or between about 240 g and about 325 g, or between about 245 g and about 320 g, or between about 245 g and about 320 g. In one embodiment, the plunger removal force is about 247 g. In another embodiment, the plunger removal force is about 318 g. By contrast, the PLAYTEX® SIMPLY GENTLE GLIDE® product was tested by the same methodology and has a retention force of about 238 g.

In addition to the necessity of having a strong retention force, it is also desirable to have a sufficient collapse force. The plunger 80 of the present disclosure is superior to prior art plungers in this regard.

In further embodiments of the present disclosure, a compact plunger (not shown) is provided, a compact plunger includes a telescoping two-piece plunger (that makes-up plunger 80). The two-piece plunger includes an inner plunger and an outer plunger. The inner plunger is typically reward of and/or nests within (telescopically) the outer plunger. The outer plunger is typically forward of and/or nests within the grip region, transition region, and/or barrel region of the applicator barrel. The two-piece plunger is selectively positionable in a non-deployed configuration and in deployed configuration.

In some embodiments, each of the insertion ends of the inner plunger and outer plunger are deformed thereby enabling retention. Optionally, the rearward finger gripping end of the inner plunger is deformed. As one retracts (or pulls rearward) on the inner plunger, the inner plunger, by way of the deformed end, locks-into place within the outer plunger by way of a friction fit and/or an interference fit. Similarly, the deformed insertion end of the outer plunger engages the interior of the applicator barrel (the barrel region, the transition region, and/or the grip region). Once the inner and outer plungers are fully-deployed and locked, the user is able to grip and push the fully-assembled plunger thereby applying force on the distal end of the pledget and causing the pledget to be ejected from the applicator.

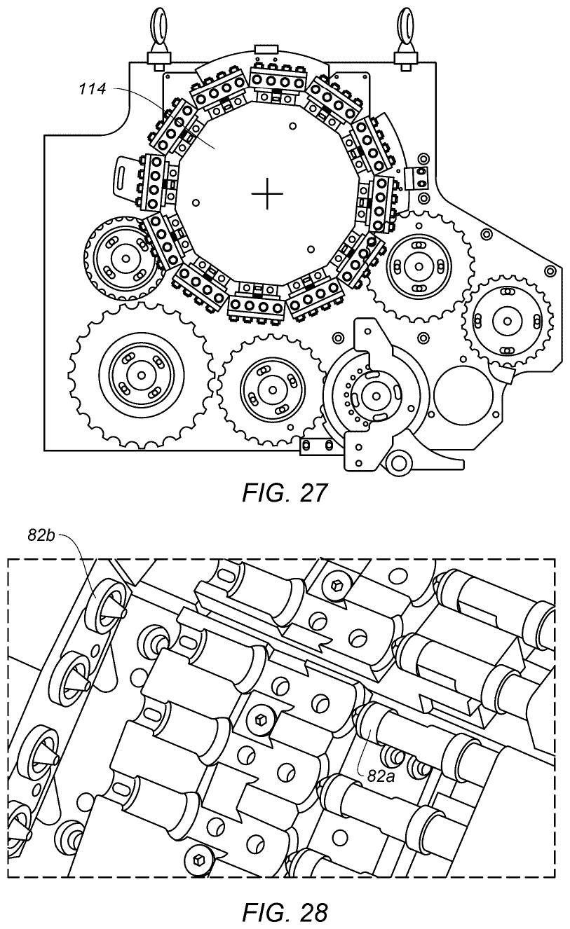

The applicator barrel 122 and plunger 80 are assembled by inserting the plunger 80 into the applicator barrel 122 either through the insertion end 130 or the plunger end 128 of grip region 132. Such assembly is achieved by way of, for instance, an HP400 machine manufactured by Hauni. For clarity, the barrel 122 is unformed (i.e., the petals 138 of the insertion end 130 have not been curved or domed and thus are straight as in FIG. 30). For clarity, the tampon 10 is not yet inserted into the applicator barrel 122. FIG. 27 is an exemplary schematic of the apparatus used to assembly the applicator barrel 122 and plunger 80, including the drums described below. FIG. 28 is a close-up schematic of an exemplary apparatus showing reshape tool 82a and 82b. The tampon applicator assembly 120 is transferred through a rotary vacuum drum 114, where the pitch diameter of the drum is about 390 mm. Other vacuum drums of different dimensions are possible but would vary cycle time. Once the applicator assembly 120 is transferred onto the main rotary vacuum drum 114, the main product carriage and the primary reshape tool 82 advances and pilots both the consumer end (which in some embodiments, is an rolled end or an inward rolled end) and the plunger 80 insertion end 108 (which is deformed into a polygonal shape). The reshape tool 82 then advances independent of the main carriage, creating the deformed end 78 on the insertion end 108 of the plunger 80, thereby improving retention and mitigating against the plunger 80 errantly falling-out of the applicator barrel 122. Optionally, the reshape tool 82 simultaneously provides either a deformed end, rolled end, or crimped end to the consumer end 110 of the plunger 80 (while providing the polygonal deformed end 78 on the insertion end 108 of the plunger 80). The plunger 80 is thus deformed (or formed, or rolled) while being telescopically engaged inside the applicator barrel 122. As such, the reshape tool 82 has an outer geometry that is smaller than the interior geometry of the applicator barrel 122. For embodiments employing a rolled 112 consumer end 110, the reshape tool 82 must be heated to at least 175 degrees Fahrenheit and more preferably greater than about 180 degrees Fahrenheit. The time required for deform, form, or roll an end 110 of the plunger 80 is roughly between about half second and about four seconds, and corresponds to the complete inwards stroke of the reshape tool 82. Once the plunger 80 has been fully formed, or deformed, or both, as the case may be, the reshape tool 82 retracts completely from the applicator barrel 122 and the carriage moves outward to pull the plunger 80 off of the roll tool so the plunger 80 can be horizontally transferred to the next and adjacent drum for the inspection process. After the inspection process is completed, the tampon applicator is loaded with the pledget and the insertion end of the applicator barrel 122 is closed with an applicator doming tool.

According to an aspect of the present disclosure, an apparatus for forming an insertion end 108 and/or consumer end 110 of a plunger 80 is provided and is referred to as the reshape tool 82. The reshape tool 82 is generally shown by exemplary FIGS. 22-28. FIGS. 25-26 include exemplary but non-limiting dimensions of a reshape tool according to the present disclosure. The reshape tool 82 includes a forming die for shaping an insertion end 108 and/or consumer end 110 of a plunger 80. In certain embodiments, there are two reshape tools 82, where a first reshape tool 82a forms the insertion end 108 into a shape other than the plunger's 80 initial unformed and generally cylindrical shape, and a second reshape tool 82b forms the consumer end 110 into a shape other than the plunger's 80 initial unformed and generally cylindrical shape, where the first and second insertion dies provide a different shaped end (and thus the first die and the second die are different).

In certain embodiments, a single reshape two 82 has two ends having separate configurations that provide two different shaped ends. In addition to the reshape tool(s) 82, the apparatus further includes the aforementioned machinery and rotating vacuum drum 114. The reshape tool 82 has an outer cylindrical portion 84 with an outer diameter 86 of between about 12 mm and about 19 mm, or between about 14 and about 18 mm, or between about 15 mm and about 17 mm, and an outer height 88 between about 3 mm and about 6 mm, or between about 3 mm and about 5 mm, or about 4.5 mm to about 5 mm.

Reshape tool 82 has adjacent and inward of the outer cylindrical portion 84 is a recessed middle portion 90 having a height 92 that is less than the outer height. The recessed middle portion 90 height 92 is between about 0.5 mm and about 4 mm, or about 0.5 mm and about 1.5 mm, or about 2.5 mm to about 3.5 mm. The recessed middle portion 90 has a width 94 between about 1 mm and about 4 mm. The recessed middle portion 90 optionally has a tapered geometry such that the upper end 118 has a greater width than a lower end 119. The lower end 119 optionally has a radius of curvature 116 of about 0.5 mm to about 1.5 mm, or about 1.0 mm. A taper angle 115 between the upper end 118 and the tapered middle portion 90 is between about 30 degrees and about 50 degrees, between about 35 degrees and about 45 degrees, or about 40 degrees.

Inward and adjacent the recessed middle portion 90 is a central portion 96 that is generally cylindrical, the central portion 96 having a tapered orientation end 98. The central portion 96 has an uppermost surface 95 with a diameter of about 3 mm to about 5 mm, or about 3.25 mm to about 4 mm. The central portion 96 has a central portion height 100 that is greater than the outer height 88, the central portion 96 having a base region 102 having a polygonal shape. The polygonal shape has at least three points 105. Each point 105 has a radius of curvature 113 of about 0.5 mm to about 1.5 mm. As the plunger 80 is pushed onto reshape tool 82, the points 105 deform the plunger end 78 into a polygonal shape with a diameter 76 greater than the maximum diameter 106 of the main body portion 104. In some embodiments such as shown in exemplary FIG. 26, lip 103 is that is about 1 mm to about 3 mm or about 1.5 to about 2 mm above base region 102. Lip 103 is a forming surface for consumer end 110.

A central taper angle 117 is defined as the angle between a vertical projection parallel to the central axis of the reshape tool 82 between the base region 102. The central taper angle 117 is between about 5 degrees and about 30 degrees, about 10 degrees and about 20 degrees, or about fifteen degrees. An upper taper angle 109 is defined as the angle between a vertical projection parallel to the central axis 99 of the reshape tool 82 and the upper sloped surface 97 is greater than the taper angle 117 to create further lead-in for the plunger. The upper taper angle 109 is between about 30 degrees to about 60 degrees, or about 40 degrees to about 50 degrees, or about 45 degree 50

As previously referred to but now in greater detail, and as exemplified in FIGS. 29-31, the applicator barrel 122 has a length 136 that extends between a plunger end 128 and an insertion end 130. The barrel 122 includes a grip region 132, a transition region 134, a main body region 136, an insertion end region 137 having a plurality of petals 138, and an interior cavity 140 extending between the insertion end 130 and the plunger end 128. The transition region 134 has a length 135 and is disposed between the main body region 136 and the grip region 132. The petals 138 are disposed between the insertion end 130 and the main body region 136. The main body region 136 extends from the petals 138 to the transition region 134, and the grip region 132 extends from the plunger end 128 to the transition region 134.

In some embodiments, the main body region 136 is defined by a circumferentially extending main body wall 142 having an outer surface 144 disposed at a constant outer diameter and an inner surface 146 disposed at a constant inner diameter; e.g., the wall 142 has a generally constant thickness of between about 0.5 mm and about 4 mm. The consumer end 110 has a wall thickness of between about 0.5 mm and about 4 mm. In some embodiments, the consumer end 110 has a wall thickness that is greater than the wall thickness of the main body region 136, particularly where the consumer end 110 is a rolled end 112. The wall thickness of the insertion end 108 is preferably between about 0.5 mm and about 4.0 mm. In some embodiments, the insertion end has about the same wall thickness as the main body region 136. The inner surface 146 having a constant inner diameter along the main body wall 142 can help create a consistent ejection of the tampon 10 into the user's body. In other words, the generally constant inner diameter helps avoid instances where the tampon 10 might wobble (i.e. if the tampon's 10 outer diameter is less than the inner diameter) as the unitary and deployed plunger 80 pushes the tampon 10 out of the barrel 122 of the applicator 120. The main body wall inner surface 146 defines a portion of the interior cavity 140 of the barrel 122. In some embodiments the main body region 136 is defined by a main body wall 142 having a tapered configuration with the outer surface 144 disposed at a first outer diameter adjacent the transition region 134 and disposed at a second outer diameter adjacent the insertion end 130 of the petals 138, with the first outer diameter greater than the second outer diameter; e.g., the main body region 136 decreasingly tapers in the direction from the transition region 134 the insertion end region 137 with petals 138, and/or decreasingly tapers in the direction opposite to the direction in which the transition region 134 tapers (i.e. the transition region 134 tapers towards the grip region 132). A barrel 122 that varies in external geometry (i.e. along the outer surface 144) can provide an improved user experience as the product is used (i.e. inserted and/or removed) from the user's body. FIGS. 29-31 illustrate a main body region 136 having a generally circular shaped cross-section. The present disclosure is not limited to a main body region 136 having a circular cross-sectional shape.

The grip region 132 is defined by a circumferentially extending grip wall 148 having an inner surface 150 disposed at a constant inner diameter and an outer surface 152. The grip wall inner surface 150 defines a portion of the interior cavity 140 of the barrel 22. The grip region 132 has a length 154 that extends between the plunger end 128 and the transition region 134, which length 154 is typical for a full-length barrel 122. In some embodiments, the outer surface 152 of the grip wall 148 may have a constant outer diameter. In some embodiments, the grip wall outer surface 152 may include protrusions 153 that extend radially outwardly from the outer surface 152; e.g., to facilitate the user's grip of the device. In some embodiments, the grip region 132 may include an outwardly flared section 156 disposed at the plunger end 128; e.g., the outwardly flared section 156 may transition from the outer diameter of the grip region 132 to a second diameter contiguous with the plunger end 128, which second diameter is greater than the outer diameter of the grip region 132. The outer diameter 174 of the flared section 156 is at least about 0.050 inches (about 1.3 cm) greater than the diameter 170 of the recessed portion of the grip region 136. Preferably, the outer diameter 174 of the flared section 56 is at least about 0.075 inches (about 0.2 cm) greater than the outer diameter 170 of the recessed portion of the grip region 36. More preferably, the outer diameter 174 of the flared section 56 is at least about 0.100 inches (about 0.2 cm) greater than the outer diameter 170 of the recessed portion of the grip region 36. In preferred embodiments, the length 154 of the grip region 132 is greater than 0.5 inches (12.7 mm). In other preferred embodiments the length 154 of the grip region 132 is also less than 0.8 inches (20.3 mm). In yet other preferred embodiments, the grip region 132 may have a varying inner diameter along the inner surface 150 and/or a varying outer diameter along the outer surface 152.

The transition region 134 is defined by a circumferentially extending transition wall 158 having an outer surface 160 and an inner surface 162. The transition wall inner surface 162 defines a portion of the interior cavity 140 of the barrel 122. The outer surface 160 is disposed at an outer diameter that changes from the grip region 132 to the main body region 136. For example, the outer diameter of the transition wall 158 at the interface with the grip region 132 may be equal to the outer diameter of the grip region outer surface 152, and may taper outwardly increasing in diameter in the direction of the main body region 136. At the interface with the main body region 36, the outer diameter of the transition wall 158 may equal the outer diameter of the main body region outer surface 144. The transition region 134 generally has a length 135 of about 0.100 inches (about 0.2 cm) to about 0.500 inches (about 1.3 cm).

The plurality of petals 138 may be integrally attached to the main body region 136 of the barrel 122. The present disclosure is not limited to any particular number of petals 138, or any particular petal configuration. In other words, the applicator of the present disclosure could have two, three, four, five, six, seven, or eight or more petals 138, depending on a plurality of factors such as the desired ejection force of the tampon 10 and/or the column strength the unitary and deployed plunger 80 can provide. In some embodiments, the petals 138 and/or insertion end region 144 in general can have an elongate or tapered geometry thereby facilitating comfortable insertion. The tampon has an insertion end 44 opposite its withdrawal end 46. In some embodiments, the tampon 10 can have an insertion end 44 that has a complimentary tapered or elongate shape. Such shapes can include elliptical, hyperbolic and/or parabolic curves such that the curve is along the profile of the petals 138 or tampon insertion end 44.

FIGS. 29-31 are exemplary embodiments of applicators having various dimensions, all of which fall within the ranges of dimensions described throughout the present disclosure. The overall barrel length 176 is about 2.85 inches (about 7.2 cm) to about 2.9 inches (about 7.4 cm), where the grip region 132 has a length 154 of about between about 0.5 inches (about 1.2 cm) to about 0.8 inches (about 2 cm), the main body region 136 has an outer diameter 172 adjacent the transition region 138 of about 0.47 inches (about 1.2 cm) to about 0.6 inches (about 1.5 cm), and a plunger end 128 outer diameter 174 of about 0.47 inches (about 1.2 cm) to about 0.65 inches (about 1.7 cm).

Optionally, the insertion end region 137 has elongated and tapered petals that fall within a Taper Ratio of about 0.3 to about 1.0. The “Taper Ratio” is defined as the radius 178 of the insertion end region 137 at the base of the petals 38 (i.e. where the cuts between each petal end/meet the main body region) divided by the length 180 of the insertion end region 137 (i.e. the petal tip ends 179 to the base 139 of the petals 138).

In some embodiments, the tampon 10 can have a reduced overall length to increase the flexibility in barrel design such that one or more full-size barrel components (or at the very least, one or more barrel components that have characteristics that more closely resemble full-size applicators than known compact applicators) can be utilized in a compact applicator form. Tampon 10 can be compressed radially and axially to be formed with a reduced length with respect to known tampons used with compact applicators. The tampon 10, despite having reduced length, achieves absorbency comparable to existing tampons in both full-size tampon applicator assemblies and compact applicator assemblies.

One skilled in the art understands the compact applicator assembly of the present disclosure can comprise a plurality of materials and aesthetics. In particular, using aesthetics to elicit distinction amongst components of the applicator system can be advantageous. For instance, in one embodiment the grip region has a distinct aesthetic and/or tactile indicators (i.e. a material, texture, color, tone, shade, luminosity, print, pattern, graphic or other visual indicators) on with respect to the rest of the applicator barrel can assist the user in identifying where and how to hold the tampon applicator. In another embodiment, one or more parts of the applicator assembly are at least partially translucent to assist the user in identifying where aspects of the applicator are (i.e. where the tampon 10 and/or tampon string are located within the applicator, and/or where the inner sleeve is positioned in a compact configuration versus a deployed configuration). In further embodiments, the inner sleeve and outer sleeve may have different visual and/or tactile indicators.

Although this invention has been shown and described with respect to the detailed embodiments thereof, it will be understood by those skilled in the art that various changes in form and detail may be made without departing from the spirit and scope of the invention.

1-34. (canceled)

35. A tampon applicator assembly comprising:
a pledget, wherein the pledget has an absorbency of between about 6 g and about 9 g and a basis weight of between about 120 gsm and about 145 gsm or the pledget has an absorbency of between about 9 g and about 12 g and a basis weight of between about 160 gsm and about 190 gsm, wherein the gram-per-gram absorbency is between about 5 g/g and about 6 g/g; and
a plunger having an insertion end and a consumer end generally opposite the insertion end, the plunger having a body portion between the insertion end and the consumer end, wherein the insertion end is deformed from a generally cylindrical shape to a polygon shape, wherein a diameter of a circumscribed circle about the polygon is between about 9.25 mm and about 10.25 mm, wherein the body portion is generally cylindrical after to the insertion end is deformed, and wherein the length of the insertion end is between about 0.5 mm and about 5 mm.

36. A regular pledget having an absorbency of between about 6 g and about 9 g, wherein the pledget has a basis weight of between about 120 gsm and about 145 gsm, and wherein the gram-per gram absorbency is between about 5 g/g and about 6 g/g.

37. The pledget according to claim 36, wherein the pledget comprises fiber, wherein the fiber is cotton or rayon or combinations thereof.

38. The pledget according to claim 37, wherein the pledget comprises 100% cotton fiber.

39. The pledget according to claim 37, wherein the pledget has a cross-pad configuration having at least two pads, wherein the at least two pads each comprise the fiber, wherein the fiber has undergone a cross-lapping to form at least two layers and has been needlepunched with about 40 punches per square centimeter to about 80 punches per square centimeter.

40. The pledget according to claim 39, wherein the at least two pads have a length between about 7.6 cm and about 12.7 cm, and width of between about 2.54 cm and about 7.6 cm.

41. The pledget according to claim 40, wherein each of the at least two pads comprises 100% cotton.

42. A super pledget having an absorbency of between about 9 g and about 12 g, wherein the pledget has a basis weight of between about 160 gsm and about 190 gsm, and wherein the gram-per-gram absorbency is between about 5 g/g and about 6 g/g.

43. The pledget according to claim 42, wherein the pledget comprises fiber, wherein the fiber is cotton or rayon or combinations thereof.

44. The pledget according to claim 43, wherein the pledget comprises 100% cotton fiber.

45. The pledget according to claim 43, wherein the pledget has a cross-pad configuration having at least two pads, wherein the at least two pads each comprise the fiber, wherein the fiber has undergone a cross-lapping to form at least two layers and has been needlepunched with at least 80 punches per square inch.

46. The pledget according to claim 45, wherein the at least two pads have a length between about 7.6 cm and about 12.7 cm, and width of between about 2.54 cm and about 7.6 cm.

47. The pledget according to claim 46, wherein each of the at least two pads comprises 100% cotton.

48. A plunger for a tampon applicator having an insertion end and a consumer end generally opposite the insertion end, the plunger having a body portion between the insertion end and the consumer end;
wherein the insertion end is deformed from a generally cylindrical shape to a polygon shape;
wherein a diameter of a circumscribed circle about the polygon is between about 9.25 mm and about 10.25 mm;
wherein the body portion is generally cylindrical after to the insertion end is deformed; and
wherein the length of the insertion end is between about 0.5 mm and about 5 mm.

49. The plunger for a tampon applicator according to claim 48, wherein the consumer end is rolled such that the thickness of the consumer end is greater than the thickness of the body portion, wherein the thickness of the consumer end is between about 0.5 mm and about 4 mm.

50. The plunger for a tampon applicator according to claim 48, wherein the insertion end has at least three vertices.

51. The plunger for a tampon applicator according to claim 50, wherein the at least three vertices each have a radius of curvature of about 0.5 mm to about 1.5 mm.
